(19) United States
(12) Patent Application Publication
(10) Pub. No.: US 2012/0164668 A1
(43) Pub. Date: Jun. 28, 2012

(54) ULTRASOUND ASSISTED IMMUNOASSAY

(75) Inventor: Peter Starokadomskyy, Dallas, TX (US)

(73) Assignee: YEDA RESEARCH AND DEVELOPMENT CO. LTD. at the Weizmann Institute of Science, Rehovot (IL)

(21) Appl. No.: 13/386,881

(22) PCT Filed: Jul. 27, 2010
(86) PCT No.: PCT/IL10/00598

§ 371 Date: Jan. 24, 2012

Related U.S. Application Data
(60) Provisional application No. 61/228,982, filed on Jul. 28, 2009.

(51) Int. Cl. G01N 33/566; B06B 1/00; G01N 33/577

(52) U.S. Cl. 435/7.92; 436/501; 422/128

(57) ABSTRACT

The invention relates to methods, devices and systems for an enhanced, ultrasound assisted immunoassay (such as a Western blot immunoassay). In particular, there are provided methods, devices and system for the automated and enhanced processing of a membrane having proteins associated therewith, wherein the processing includes the use of ultrasonic energy.

### FIELD OF THE INVENTION

The present invention generally relates to methods, device and system for an enhanced ultrasound assisted immunoassay, such as, for example, a Western blot immunoassay. In particular, there are provided methods, devices and system for the automated and enhanced processing of a membrane having proteins associated therewith, wherein the processing includes the use of ultrasonic energy.

### BACKGROUND

Immunoassay methods are used for the identification and quantitation of a substance (antigen) of interest, in a biological sample, based on the interaction between the substance of interest (the antigen) and its respective antibody. The antigen may include various substances, such as, for example, a chemical molecule, a drug, a protein, a peptide, a microorganism (such as, for example, a virus), nucleic acids (such as, for example, DNA and RNA), and the like. The antibody-antigen interaction may be further identified, measured and/or quantified by various means, either directly or indirectly, and may include the use of, for example, labeling the antigen and/or the antibody. The labeling may include any type of labeling that may be detected, identified, measured and/or quantified. For example, the labeling may include the use of colloidal gold; radioisotopes, such as, for example, I-125, P-32, C-14, H-3; magnetic labels, fluorescence labels; chemiluminescence labels; enzymes; additional antibodies (secondary antibodies); and the like. Some of the immunoassays known in the art include such methods as, Enzyme linked immunosorbent assay (Elisa), Radio immuno assay (RIA), Western Blot, Far eastern blot, immunohystochemistry, immunocytochemsitry, agglutination, nephelometry, immunoprecipitation (IP), and the like.

Western blot immuno assay analysis is generally used to detect a protein of interest (an antigen) in a biological sample (that may include, for example, a mixture of any number of proteins), while providing information about the size and amount of the protein of interest. The Western blot method usually involves the separation of the protein of interest from other proteins and substances in the examined sample, by means of a gel (such as, for example, a polyacrylamide gel), which is used to separate the proteins according to, for example, their weight. For example, in polyacrylamide gel electrophoresis (PAGE), charged proteins are separated in polyacrylamide gels based on their size (molecular mass) in native and denatured form. After the gel separation, the proteins resolved in the gel are transferred to a membrane (such as, for example, nitrocellulose, nylon, polyvinylidene fluoride polyvinylidene difluoride (PVDF) membranes, and the like). The presence of the protein of interest may then be detected by antibodies specific to the protein of interest, which are in turn detected by antibody-binding reagents. Antibody-binding reagents may include, for example, Protein A, Protein G, or secondary antibodies, which may be radiolabeled, enzyme-linked, gold labeled, magnetically labeled, and the like, to allow their visual detection and optionally to further allow their quantitation. The detection itself may be performed by various means, such as, for example, by autoradiography, colorimetric reaction, chemiluminescence, and the like. Furthermore, the use of specific antibodies against various protein modifications (such as, for example, phosphorylation, ubiquitilation, and the like) may further be used to provide valuable information about molecular mechanisms in which the protein of interest is involved.

Western blot analysis as performed today has several disadvantages, which are dependent on the reagents used in the process. First, it is dependent on the use of a high-quality antibody directed against the protein of interest. Antibodies are the most expensive reagent, and its quality is critical for getting objective results. Secondly—difference between levels of target bands (which correspond to the protein of interest) and background level is critical for detection of weak signals. Removal of the background is usually performed by numerous washing steps, aimed to lower the background levels as much as possible. However, the washing steps are time consuming and may extend the analysis process by at least 25%. Third, processing and developing the membranes onto which the proteins from the gel has been transferred, are performed manually. The various processing steps, such as, for example, stripping, washing, blocking, incubation with antibodies, and the like, usually involve placing the membrane in different solutions for varying period of time. These processing steps are time consuming and include exhausting and repetitive work, which may usually lead to errors that may result in inconsistency and inaccuracy of the results, increased processing time, different saturation of target band or background, and the like. Moreover, when simultaneously developing several membranes, value of errors may accumulate and increase with the amount of assays.

It has been previously described that use of ultrasonic irradiation at various frequency and power ranges, may influence antibody-antigen interactions and may thus be used in various immunoassays. For example, a publication by Haga et.al. (1987), entitled “Effect of ultrasonic irradiation on the dissociation of Antigen-Antibody Complexes. Application to homogenous Enzyme Immunoassay”, studied the effect of ultrasonic irradiation on the dissociation of antigen-antibody complexes. A publication by Chen et.al. (1984) entitled “Ultrasound-accelerated immunoassay, as exemplified by enzyme immunoassay of choriogonadotropin”, is directed to the study of the influence of ultrasound on the kinetics of antigen-antibody interactions in solid-phase immunoassays. A publication by Nelson, et.al. (2006), entitled “Protocol for the fast chromatin immunoprecipitation (ChIP) method”, discloses the use of an ultrasonic bath to increase the rate of protein-antibody binding. US Patent Application No. US 2009/0053688 discloses method and device for ultrasound assisted particle agglutination assay. U.S. Pat. No. 7,090,974, discloses ultrasound mediated high speed biological reaction and tissue processing and is directed toward a method of decreasing the time for conducting histology or pathology study no tissue samples, e.g., biological reactions, fixation, processing, embedding, deparaffinizing, and dehydration by applying ultrasound to the tissue during these processes. However, none of the above references disclose nor suggest the use of ultrasonic energy in the steps of the methods, device and/or system described herein.

Hence, there is a need for a method and device that would enhance the Western blot immuno assay by shortening the overall length of the Western Blot analysis procedure in general, and in particular by reducing manual hand-on time, in order to obtain faster, more accurate, more reliable and higher quality results. This will further allow saving time and expensive reagents, as well as allow the standardizing of the Western blot procedure.

### SUMMARY

The following embodiments and aspects thereof are described and illustrated in conjunction with systems, devices and methods which are meant to be exemplary and illustrative, not limiting in scope.

According to some embodiments, the present invention provides methods, device and system for ultrasound assisted immunoassays (such as western blot immunoassay), for the enhancement of such assays.

According to some embodiments of the present invention there is provided a method for enhancing an immunoassay for identification of proteins associated with a membrane, wherein the method includes the use of ultrasonic energy during washing steps of the method. There is further provided a device and system for the automated processing of a membrane having proteins associated therewith. The device and system include one or more ultrasonic transducers adapted to emit ultrasonic energy at the membrane at various washing steps during the processing of the membrane.

According to some embodiments, there is provided a method for enhanced immunoassay for identification of proteins which includes separating the proteins by electrophoresis in a porous matrix; electrophoretically transferring the proteins from the porous matrix to a membrane; contacting the membrane with one or more antibody solutions for a period of time; and washing the one or more antibody solutions from the membrane in the presence of ultrasonic energy directed at the membrane.

According to other embodiments, there is provided a device for processing of a membrane having proteins associated therewith, the device includes a container situated on a platform, wherein the container is adapted to retain the membrane; and an ultrasonic transducer, adapted to emit ultrasonic energy at the membrane retained within the container.

According to some embodiments, there is provided a method for the enhanced immunoassay for identification of proteins, the method includes: separating the proteins by electrophoresis in a porous matrix; electophoretically transferring the proteins from the porous matrix to a membrane; contacting the membrane with one or more antibody solutions for a period of time; and washing the one or more antibody solutions from the membrane in the presence of ultrasonic energy directed at the membrane.

According to some embodiments, there is provided a method for processing a membrane having proteins associated therewith, the method includes contacting the membrane with one or more antibody solutions for a period of time; and washing the one or more antibody solutions from the membrane in the presence of ultrasonic energy directed at the membrane.

According to further embodiments, the porous matrix includes a polyacrylamide gel, an agarose gel, gelatin gel, or any combination thereof. The membrane includes nitrocellulose membrane, polyvinylidene fluoride polyvinylidene difluoride (PVDF) membrane, nylon membrane, or any combination thereof.

According to additional embodiments, the antibody solution includes a polyclonal antibody, a monoclonal antibody, serum, conjugated antibody, or any combination thereof. The period of time over which the antibody solution is contacted with the membrane is in the range of 10 minutes to 24 hours.

According to some embodiments, the washing step may be repeated one or more times at equal or different time intervals.

According to further embodiments, the ultrasonic energy may be applied by one or more ultrasonic transducers. The one or more ultrasonic transducers are adapted to emit ultrasonic energy at a frequency of 0.1-1000 kHz.

According to further embodiments, the method may further include exposing the washed membrane to a photo-sensitive film.

According to some embodiments, there is provided a device for processing of a membrane having proteins associated therewith, said device includes a container situated on a platform, wherein the container is adapted to retain the membrane; and an ultrasonic transducer, adapted to emit ultrasonic energy towards the container.

According to additional embodiments the processing includes: washing, incubating, shaking, rocking, developing, applying ultrasonic energy or any combination thereof.

According to further embodiments, the membrane comprises nitrocellulose, polyvinylidene fluoride polyvinylidene difluoride (PVDF), nylon or any combination thereof. The proteins may be electrotransferred to the membrane.

According to further embodiments, the device may further include a control unit. The control unit may include a user interface, a display, a key pad, an alarm, a dial, or any combination thereof. In some embodiment, the platform of the device may be attached to a motor. The platform may be adapted to move horizontally and/or vertically around an axis. The platform may be adapted to move at constant speed, varying speed, or both. The speed may be in the range of 0 rpm to 1000 rpm.

According to further embodiments, the ultrasonic transducer may be adapted to emit ultrasonic energy at a frequency of 1-1000 kHz. The ultrasonic transducer may be located above the container. The ultrasonic transducer may be located below the container. The ultrasonic energy may be focused ultrasonic energy. The ultrasonic energy may be a surface ultrasonic energy. In some embodiments, the ultrasonic transducer may include more than one oscillating elements.

According to additional embodiments, there is provided a system for the automatic processing of a membrane having proteins electrophoreted thereto, the system comprises: a container situated on a platform, wherein the container is adapted to retain the membrane; an ultrasonic oscillator, adapted to emit ultrasonic energy towards the container; and a pump, adapted to add or remove fluid to or from the container from one or more tanks. In some embodiments, the processing includes washing, incubating, shaking, rocking, developing, applying ultrasonic energy or any combination thereof. The membrane may include nitrocellulose membrane, polyvinylidene fluoride polyvinylidene difluoride (PVDF) membrane, nylon membrane, or any combination thereof. The fluid may include water, saline, buffer, PBS, TBS, blocking solution, milk, BSA solution, antibody solution, or any combination thereof.

According to further embodiments, the system may further include a central control unit. The central control unit may include a user interface, a display, a key pad, an alarm, a dial, or any combination thereof. In some embodiment, the platform of the system may be attached to a motor. The platform may be adapted to move horizontally and/or vertically around an axis. The platform may be adapted to move at constant speed, varying speed, or both. The speed may be in the range of 0 rpm to 1000 rpm. According to further embodiments, the ultrasonic transducer may be adapted to emit ultrasonic energy at a frequency of 1-1000 kHz. The ultrasonic transducer may be located above the container. The ultrasonic transducer may be located below the container. The ultrasonic energy may be focused ultrasonic energy. The ultrasonic energy is surface ultrasonic energy. In some embodiments, the ultrasonic transducer may include more than one oscillating elements.

According to further embodiments, the pump of the system may include peristaltic pump, mini pump, unidirectional pump, bidirectional pump, or any combination thereof. The system may further include tubes adapted to transfer fluids. In some embodiments, the tank may include a vessel, a flask, a sink, a test tube, a vial, or any combination thereof.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The present invention will be understood and appreciated more fully from the following detailed description taken in conjunction with the appended drawings. Dimensions of components and features shown in the figures are generally chosen for convenience and clarity of presentation and are not necessarily shown to scale. It is intended that the embodiments and figures disclosed herein are to be considered illustrative rather than restrictive.

FIG. 1: A flow chart of the steps of a currently practiced general western blot method;

FIG. 2: A flow chart of the steps of an enhanced immunoassay method, according to some embodiments;

FIG. 3: Schematic illustration of exemplary ultrasonic transducers, according to some embodiments;

FIG. 4: Schematic illustration of a perspective view of a device, according to some embodiments;

FIG. 5: Schematic illustration of a perspective view of a device, according to some embodiments;

FIG. 6: Schematic illustration of a perspective view of a system, according to some embodiments;

FIG. 7: A flow chart of the steps of an enhanced and automatic immunoassay method, according to some embodiments;

FIG. 8: Pictograms of immunoblot membranes processed in the presence of ultrasound at various steps of the method; and

FIG. 9: Pictograms of immunoblot membranes processed in the presence of ultrasound (A) or in the absence of ultrasound (B).

### DETAILED DESCRIPTION

The present invention provides a device, system and methods for an improved and enhanced immuno assay, such as, for example, western blot immunoassay, by employing ultrasonic energy at various steps of the methods.

The present invention is based in part on the unexpected finding that the use of ultrasonic energy at various defined steps of processing of a membrane having proteins associated therewith (such as, for example, a western blot immunoassay membrane having proteins electrotransferred thereto), results in improvement of the western blot method by obtaining faster, more accurate, more reliable and higher quality results.

As referred to herein, the terms “enhanced”, “enhancing” and “improved” in relation to an immunoassay method are directed to a faster assay, a more accurate assay, a more reliable assay, a better quality assay, a more reproducible assay, and the like, or any combination thereof. The terms “enhanced”, “enhancing” and “improved” in relation to an immunoassay method are used to compare between the existing immunoassay, as generally performed in the art, and the immunoassay as performed according to embodiments of the present invention.

As referred to herein, the term “protocol” in relation to a method or an assay is directed to the various steps performed in the method or the assay. For example, the protocol may list the action performed at each step, the order in which the steps are performed, the time length of the steps, the reagents used in the steps, repetition of the steps, and the like, or any combinations thereof.

As referred to herein, the term “ultrasound”, “ultrasonic energy”, “acoustic energy”, may interchangeably be used and are directed to any type of ultrasonic energy (such as, for example, high intensity ultrasound, low intensity ultrasound, focused ultrasound, surface ultrasound, and the like), at any frequency of about 0.1-50 MHz and/or at any energy output of about 0.1-3000 Joules, and/or at any power output of about 0.1-2000 Watt. For example, the ultrasonic energy may be in the range of about 20 KHz. For example, the power output may be about 25W. The ultrasonic energy may be produced by any type of ultrasonic transducer, such as, for example, ultrasonic generator, ultrasonic oscillator, sonicator, and the like. For example, the ultrasonic transducer may include one or more piezoelectric elements (such as for example, ceramics, metal, lead zicronate titanium, and the like), that may be used to produce acoustic waves in response to electrical energy stimulation. The shape, size, thickness, composition and spatial location of the transducing element(s) may be adjusted so as to produce a requested acoustic energy and to target the ultrasonic energy to a desired target area. In some embodiments the ultrasonic transducer may have more than one oscillating elements, adapted to produce ultrasonic energy. The oscillating elements may be identical or different in shape, size, composition and function.

As referred to herein, the term “membrane having proteins associated therewith”, relates to any type of membrane (such as, for example, PVDF membrane, nitrocellulose membrane, nylon membrane) which is in interaction with one or more proteins. The interaction may include any type of interaction between the proteins and the membrane, such as, for example, covalent interaction, transient interaction, interaction of varying strength, chemical interaction, electrostatic interaction, and the like. For example, the interaction may be between the proteins and a surface of the membrane. For example, the proteins may be integrated within the membrane. For example, the proteins may be embedded within the membrane. For example, the proteins may be attached to the membrane. In some embodiments, the proteins may be electrotransferred to the membrane.

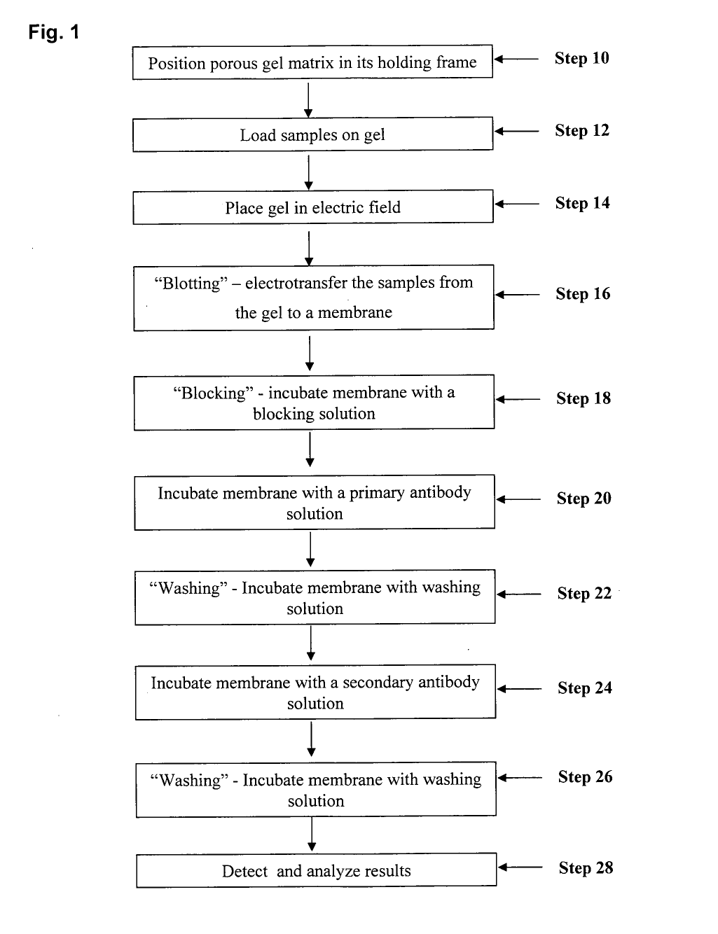

Reference is now made to FIG. 1, which illustrates a flow chart of a generally used protocol (as generally performed in the art) for western blot immunoassay. As shown in FIG. 1, the general western blot protocol includes in step 10, placing/positioning/pouring/casting a porous gel matrix (such as, for example, a polyacrylamide gel, agarose gel, gelatin gel, and the like, at various compositions and percentages) in its holding frame. The gel may be pre-casted or freshly casted into the frame. In step 12, various samples are loaded onto the gel. The samples may include a mixture of proteins, cell protein lysate, a protein marker, a known reference protein, and the like, or combinations thereof. The protein samples loaded on the gel may be denaturated or native. In step 14, the gel is placed in an electrical field (at specified current and/or voltage), for a desired period of time, and as a result, the various proteins in the samples are electrophoreted (migrating in the gel), according to their weight (which is proportional to their electrical charge). Next, in step 16 (“blotting” step) the samples are electrotransferred from the gel to a membrane under a specified voltage and/or current for a desired period of time. The membrane may include such membranes as, but not limited to PVDF membrane, Nitrocellulose membrane, nylon membrane, and the like. After the protein samples are electrotransferred to the membrane, in step 18 (the “Blocking” step), the membrane is incubated with a blocking solution for a desired period of time, such as, for example, in the range of 0.5 hours to overnight incubation. The blocking solution may include, for example, blocking proteins, such as, for example, bovine serum albumin (BSA), milk proteins, and the like, dissolved/diluted in various buffers, such as, for example, phosphate buffer solution (PBS), Tris-Buffered Saline (TBS), various detergents (such as, for example, polysorbate 20 (Tween 20)), and the like. Next, in step 20, the membrane is incubated with a primary (first) antibody solution for a desired period of time, such as, for example, about 1 hour to over-night incubation. In step 22, the membrane is washed in a washing solution (such as, for example, PBS, TBS, Tween 20, and the like), for a desired period of time, such as, for example, 5-20 minutes. Washing step 22 may be repeated any number of times, such as, for example, 3-5 times. In step 24, the membrane is incubated with a secondary antibody solution for a desired period of time, such as, for example, about 30 minutes to 4 hours. In step 26, the membrane is washed in a washing solution (such as, for example, PBS, TBS, Tween 20, and the like), for a desired period of time, such as, for example, 5-20 minutes. Washing step 26 may be repeated any number of times, such as, for example, 3-5 times. Next, at step 28, a detection reaction (such as, for example, an enhanced chemiluminescence (ECL) reaction) is performed on the membrane and the membrane is exposed to an energy sensitive substrate (such as, for example, an X-ray film, CCD camera, and the like), and the results are visualized and optionally quantified. Optionally, each of the steps in the method may be performed at a temperature ranging from 0° C. to room temperature.

The currently used western blot protocol, as outlined above, imposes several drawbacks and limitations: 1. difference between levels of target bands (which correspond to the protein of interest) and background level is critical for detection of weak signals. Removal of the background may usually be performed by numerous washing steps, aimed to lower the background levels as much as possible. However, the washing steps are time consuming and may extend the assay by at least 25%. 2. Processing and developing the blotted membranes are performed manually. The various processing steps involve placing the membrane in different solutions for varying period of time. These processing steps are time consuming and include exhausting and repetitive work, which may sometimes lead to errors that may result in inconsistency and inaccuracy of the results, increased processing time, different saturation of target band or background, and the like. In addition, simultaneous development of several membranes causes error values to accumulate and increase with the amount of assays. Thus, there is a need to improve the results obtained from the Western blot assay (by, for example, lowering background levels and increasing levels of protein of interest), make the assay more accurate and repeatable, and reduce the length of time needed to complete the assay and to make the assay much less time consuming.

According to some embodiments, there is thus provided a method of enhancing an immunoassay (such as, for example, western blot immunoassay), the method includes utilizing ultrasonic radiation at specific steps of the immunoassay method. As detailed above, it is known in the art the ultrasonic energy may accelerate antibodies-antigens interaction. To this aim, ultrasonic energy may be used for immunoassays, such as, western blot protocols, in order to remove background signals from the membrane and reduce processing time. Without wishing to be bound to any theory or mechanism, mechanical oscillation caused by the ultrasonic energy may cause detaching of non-specifically bounded antibodies because of weak force of interactions. At the same time strong complementary interaction of antibodies with their antigens would be indestructible.

According to some embodiments, there is provided a method of enhancing a western blot immunoassay, the method includes performing western blot according to a routinely used protocol and in addition, applying ultrasonic energy at various steps of the method. According to some embodiments, application of the ultrasonic energy may be performed after the blotting step (step 16 in FIG. 1), which includes electro-transferring of the electrophoreted proteins from the gel to a membrane. Application of the ultrasonic energy may be performed, for example, during the blocking step, during the incubation step with the first antibody, incubation step with the second antibody, during the washing steps, or any combination thereof.

According to some exemplary embodiments, application of the ultrasound energy may be performed during the washing steps of the membrane. The washing steps include washing the membranes with a washing buffer (such as, for example, PBS, TBS, Water, with or without additional detergents, such as, for example, Tween-20), after the incubation of the membrane with an antibody. The use of the ultrasonic treatment during the washing steps may allow decreasing the time of the washing steps by at least 3 fold. For example, the washing step without ultrasonic treatment may take between 5-10 minutes, while the use of ultrasonic treatment decreases the washing step length to about 2-3 minutes. In addition, the use of the ultrasonic energy during the washing steps allows lowering the repetition number of each washing step. For example, instead of 3 washing steps after each antibody treatment (first antibody treatment and second antibody treatment), only 1-2 repetitions of each of the washing steps are needed to achieve enhanced results (with respect to quality of the results) as compared to higher number of repetitions of the washing steps, when ultrasonic treatment is not used. Altogether, the use of the ultrasonic treatment during the washing steps allows to save over 25% of processing time and to achieve enhanced and better quality results.

According to some embodiments, the ultrasonic energy used in the western blot immunoassay method may include any type of ultrasonic energy at any frequency range (such as, for example, at the range of 1-1000 kHz), at any power (such as, for example, at the range of about 1-100 Watt), and for any time period (such as, for example 1 minute up to 2 hours). The ultrasonic energy may be focused at the membrane. The ultrasonic energy may be applied such that the entire surface area of the membrane is absorbing substantially the same amount of ultrasonic energy. The ultrasonic energy may be applied such that it is applied at the container in which the membrane is placed. The ultrasonic energy may be applied such that is aimed at a region of the container in which the membrane is placed.

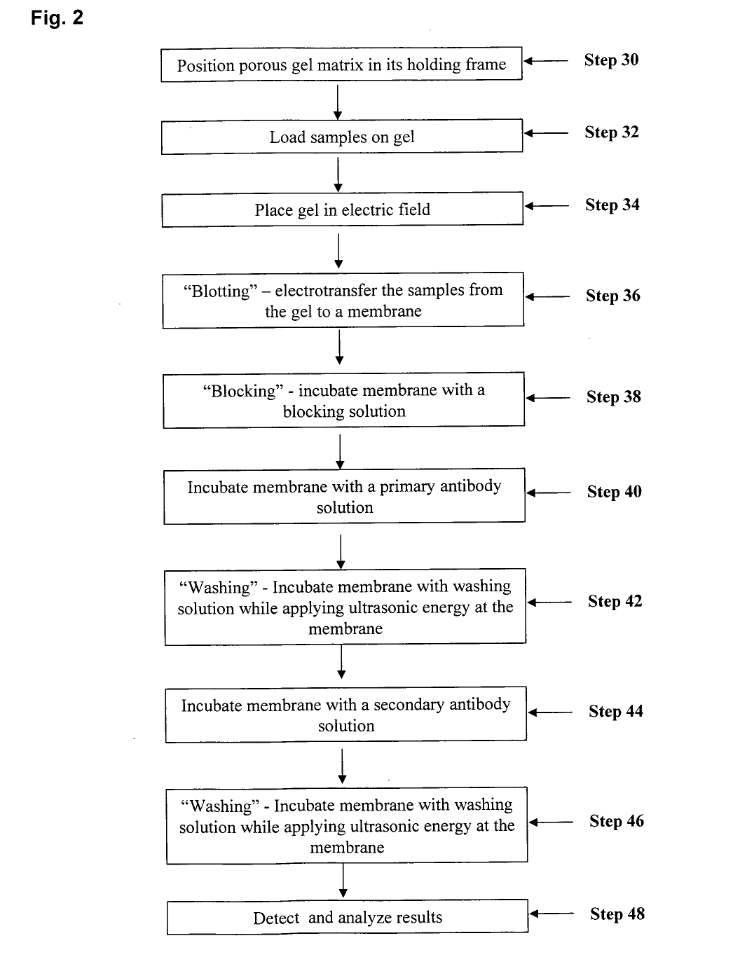

Reference is now to FIG. 2, which illustrates a flow chart of a method for an enhanced western blot immunoassay. As shown in FIG. 2, the enhanced western blot protocol includes in step 30, placing/pouring/positioning/casting a porous gel matrix (such as, for example, a polyacrylamide gel, agarose gel, and the like, at various compositions and percentages) in its holding frame. The gel may be pre-casted or freshly casted into the frame. In step 32, various samples are loaded onto the gel. The samples may include a mixture of proteins, cell protein lysate, a protein marker, a known reference protein, and the like, or any combination thereof. The protein samples loaded on the gel may be denaturated or native. In step 34, the gel may be placed in an electrical field (at specified current and/or voltage), for a desired period of time, and as a result, the various proteins in the samples are electrophoreted (migrating in the gel), according to their weight (which is proportional to their charge). Next, at step 36 (“blotting” step), the samples are electrotransferred from the gel to a membrane at a specified voltage and/or current for a desired period of time. The membrane may include such membranes as, but not limited to PVDF membrane, Nitrocellulose membrane, nylon membrane, and the like. After the protein samples are electrotransferred to the membrane, in step 38 (the “Blocking” step), the membrane may be incubated with a blocking solution for a desired period of time, such as, for example, in the range of 0.5 hours to overnight incubation. The blocking solution may include, for example, blocking proteins, such as, for example, bovine serum albumin (BSA), milk proteins, and the like, dissolved/diluted in various buffers, such as, for example, PBS, TBS, water, various detergents, and the like. Next, in step 40, the membrane is incubated with a primary antibody solution for a desired period of time, such as, for example, about 1 hour to over-night incubation. In step 42, the membrane is washed in a washing solution (such as, for example, PBS, TBS, water, Tween-20, and the like), for a desired period of time, such as, for example, 1-3 minutes, while ultrasonic energy produced by an ultrasonic transducer is applied at the membrane. The ultrasonic energy may be, for example at the frequency of 1-1000 kHz, at a power of 1-100 Watt. Washing step 42 may be repeated any number of times, such as, for example, one more time. In step 44, the membrane is incubated with a secondary antibody solution for a desired period of time, such as, for example, about 30 minutes to 4 hours. In step 46, the membrane is washed in a washing solution (such as, for example, PBS, TBS, water, Tween20, and the like), for a desired period of time, such as, for example, 1-3 minutes, while ultrasonic energy produced by an ultrasonic transducer is applied at the membrane. The ultrasonic energy may be the same or different as the ultrasonic energy applied at step 42. Washing step 46 may be repeated any number of times, such as, for example, one more time. Next, at step 48, a detection reaction (such as, for example, an enhanced chemiluminescence (ECL) reaction) is performed on the membrane and the membrane is exposed to an energy sensitive substrate (such as, for example, an X-ray film, CCD camera, and the like) and the results are visualized and optionally quantified. Optionally, each of the steps in the method may be performed at a temperature ranging from 0° C. to room temperature, for example, at 4° C.

According to some embodiments, there is provided a method for performing an enhanced/improved immunoassay for identification of proteins, wherein the enhancement/improvement is washing one or more antibody solutions from the membrane in the presence of ultrasonic energy directed at the membrane.

According to further embodiments, the method for enhanced immunoassay may be used to enhance further manipulation and reuse of the blotted membranes. As known in the art, an already blotted membrane may be reused many times. After the detection reaction (for example, steps 28 and 48 in FIGS. 1 and 2, respectively), the membrane may be “stripped” and be reused many times, in a method known in the art as “stripping”, which includes incubating the membrane with various solutions that are used to “strip” the membrane of any bound antibodies. Following standard stripping protocols, after each stripping, the membrane is incubated with a blocking solution (such as, for example, BSA and/or non-fat milk solutions). In contrast, it was surprisingly found that for membranes that are processed according to methods for enhanced western blotting immunoassay, a repeated blocking step after stripping is not required. It has been found that omitting of blocking steps after the stripping protocol does not affect the quality of the results obtained. This results in additional time saving, since omitting of blocking steps after each striping of the membrane, allows saving at least 25% of the processing time.

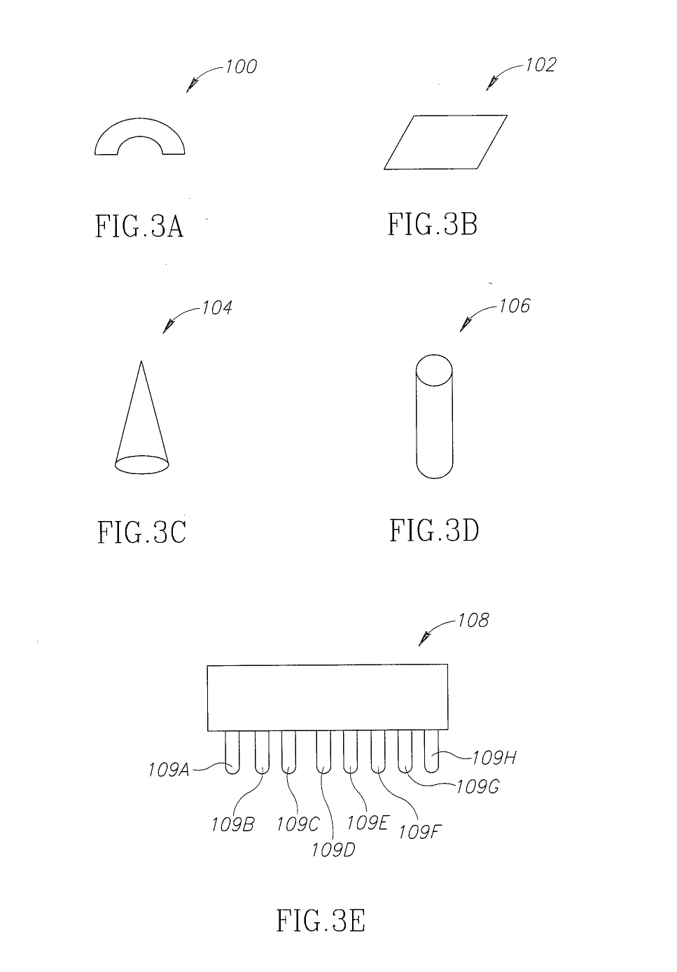

According to some embodiments, the ultrasonic energy may be applied by any type of ultrasonic energy source, such as, for example, ultrasonic transducer, ultrasonic oscillator, sonicator, and the like. For example, the ultrasonic transducer may include one or more piezoelectric elements (such, ceramics, metal, lead zicronate titanium, and the like), that may be used to produce acoustic waves in response to electrical energy stimulation. The shape, size, thickness, composition and spatial location of the transducing element(s) may be adjusted so as to produce a requested acoustic energy and to target the ultrasonic energy to a desired target area. The ultrasonic transducer may include one or more oscillating elements that may be identical or different in size, shape, structure, function and/or composition. If more than one oscillating elements is used, the elements may be operated simultaneously at the same time, or at different times. If more than one oscillating elements is used, the elements may be operated with the same or different operating parameters. The ultrasonic transducer may be or may not be in direct contact with the buffer in which the membrane is placed in. The ultrasonic transducer may be or may not be in direct contact with the membrane. The ultrasonic transducer may be used to apply focused and/or surface ultrasonic energy towards the membrane. The ultrasonic transducer may be automatically and/or manually operated at various operating parameters that may be predetermined and/or determined manually by the user. Reference is now made to FIG. 3, which schematically illustrates exemplary ultrasonic transducers that may be used to produce ultrasonic energy for use in the method of enhanced immunoassay, according to some embodiments. As shown in FIG. 3A, the ultrasonic transducer (100) may have a concave shape that may be used to direct focused ultrasonic energy. As shown in FIG. 3B, the ultrasonic transducer (102) may have a flat shape. As shown in FIG. 3C, the ultrasonic transducer (104) may include a cone shape. As shown in FIG. 3D, the ultrasonic transducer (106) may include a cylindrical shape. The ultrasonic transducer may include one or more oscillating elements that may be identical or different in size, shape, structure and/or composition. For example, as shown in FIG. 3E, the ultrasonic transducer (108) may include eight oscillating elements (109A-H), the oscillating elements may be identical or different in size, composition and shape.

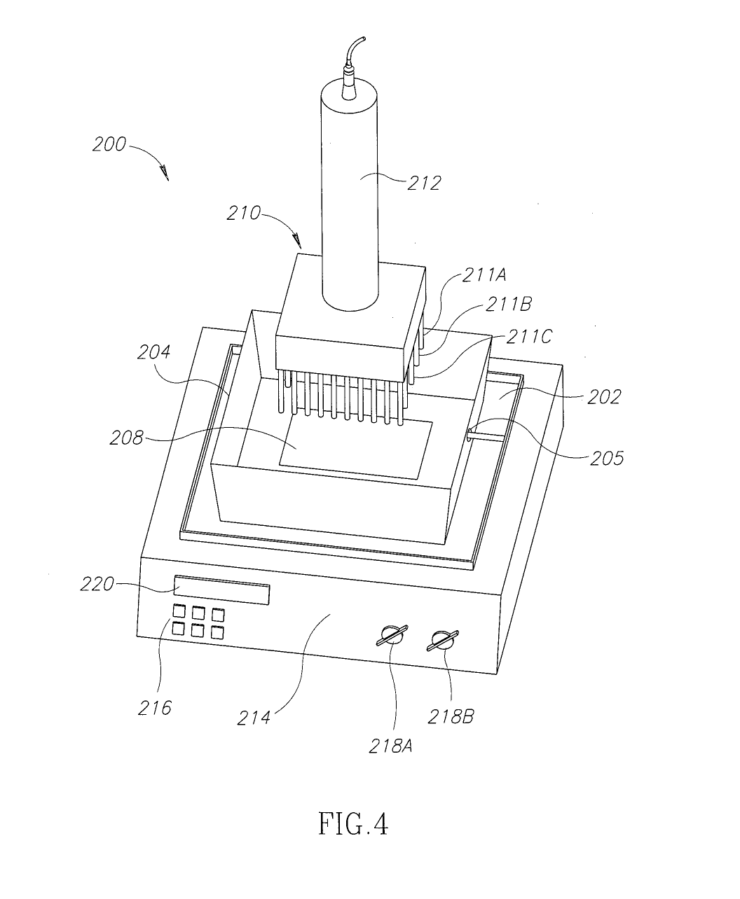

According to further embodiments, there is provided a device and system for the automatic and enhanced processing of membranes having proteins associated therewith. Reference is now made to FIG. 4, which illustrates a block diagram of a device for the enhanced processing of membranes having proteins associated therewith, according to some embodiments. The device (200) may include one or more shaking platforms (such as platform 202), which are attached to a motor (not shown). The shaking platform may move horizontally and/or vertically around an axis, at constant speed, varying speed, or both, wherein the speed at which the shaking platform is moving may be in the range of, for example, 0 rpm to 1000 rpm and may be automatically and/or manually determined. In some embodiments, the shaking platform may be also function as a heating/cooling platform. On the top surface of the shaking platform one or more containers (such as container 204) may be placed. The container may be at any shape and size. The container may have rigid walls and may be constructed of plastic, glass, and the like. The container is constructed such that it may be used to hold/retain fluids. Optionally, container 204 may have one or more openings/aperture (such as aperture 205), which may be used to allow at will the removal or addition of fluids to the container. Optionally container 204 may include a lid. Within container 204, a membrane having proteins associated therewith (such as membrane 208) may be placed. An ultrasonic transducer (such as transducer 210) is located such that ultrasonic energy produced by the transducer is directed towards container 204. The ultrasonic transducer may include any type of ultrasonic transducer. The ultrasonic transducer may include one or more oscillating elements. For example, in FIG. 4, transducer 210 includes 96 oscillating elements, of which indicated are oscillating elements 211A-C. The oscillating elements may identical or different in size, form and function. The ultrasonic transducer may produce ultrasonic energy at the range of about 1-1000 kHz, at the power of about 1-100 Watt. The ultrasonic transducer may located be at any vertical distance from the container. For example, the ultrasonic transducer may be located above the container. For example, the ultrasonic transducer may be located below the container. The ultrasonic transducer may be in direct contact with the fluids in the container. The ultrasonic transducer may be controlled by the transducer control unit (such as transducer control unit 212 which is adapted to provide the ultrasonic transducer with the appropriate operating parameters (such as, for example, power, frequency, pulse length, and the like). In addition, the device may include a controller unit (such as controller unit 214), which may include a user input unit (such as keypad (216), keyboard, dials (218A-B), buttons, touch screen, and the like, or any combination thereof), a display (220), an alarm sub-unit, and the like. The controller unit may be used to control the operation of the device. For example, the controller unit may be used to set the speed, direction and length of time at which the shaking platform is moving. For example, the controller unit (214) may be used to control the ultrasonic transducer control unit (212).

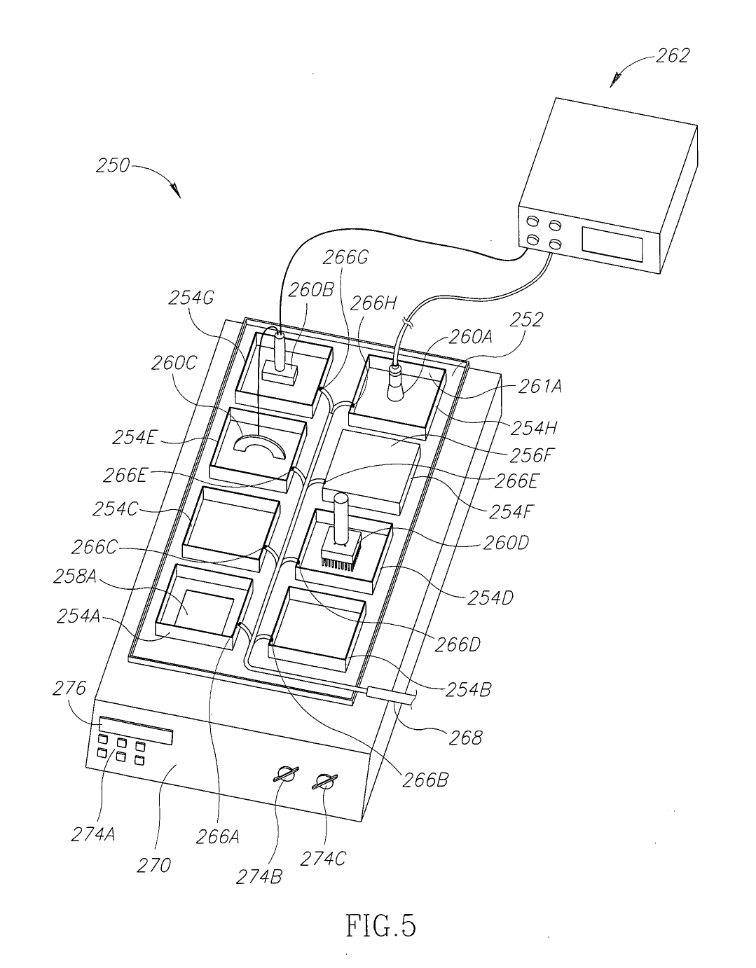

Reference is now made to FIG. 5, which schematically illustrates a perspective view of a device, according to some embodiments. As shown in FIG. 5, device 250, includes a shaking platform, 252, which may also function as a cooling/heating surface. On the shaking platform, containers, such as containers 254A-H are placed. The containers may have lids (such as lid 256F). In the containers, membranes having proteins associated therewith, such as, for example, membrane 258A, may be placed. In addition, one or more ultrasonic transducers, such as, for example, ultrasonic transducers 260A-D are located such that ultrasonic energy produced by the transducers is directed towards the respective containers (such as containers 254A-H). The ultrasonic transducers (such as ultrasonic transducers 260A-D) may be located above the containers. The ultrasonic transducers may be attached permanently or reversibly to the containers lid (such as, for example, transducer 260A and Lid 261A). The ultrasonic transducers may be in contact with fluids retained within the container. The ultrasonic transducer may include more than one oscillating elements, such as shown, for example, exemplary ultrasonic transducer 260D includes 96 oscillating elements. The ultrasonic transducers may be located below the containers (not shown). The ultrasonic transducer may be connected to an ultrasonic transducer control unit (ultrasonic transducer controlling unit 262) that may provide the ultrasonic transducers power and other operating parameters. The ultrasonic transducers may each have a separate ultrasonic transducer controlling unit or may be connected to a common ultrasonic transducer control unit. The ultrasonic transducers may be identical or different in size, shape, composition and function. The ultrasonic transducers may operate at the same or different time at the same or different operating parameters. In addition, shown in FIG. 5, are openings/apertures 266A-H located at the side walls of containers 254A-H. The apertures allow the passive or active transfer of fluids to and from the containers, for example, by connecting to tubes, such as fluid tube 268. Also shown is controller unit 270, which may include a user input unit (which includes, for example, keypad (274A), dials (274B-C)), a display (276), an alarm sub-unit, and the like. Controller unit 270 may be used to control the operation of the device (250). For example, the controller unit may be used to set the speed, direction and length of time at which the shaking platform (252) is moving. For example, the controller unit (270) may be used to control the ultrasonic transducer controlling unit (262).

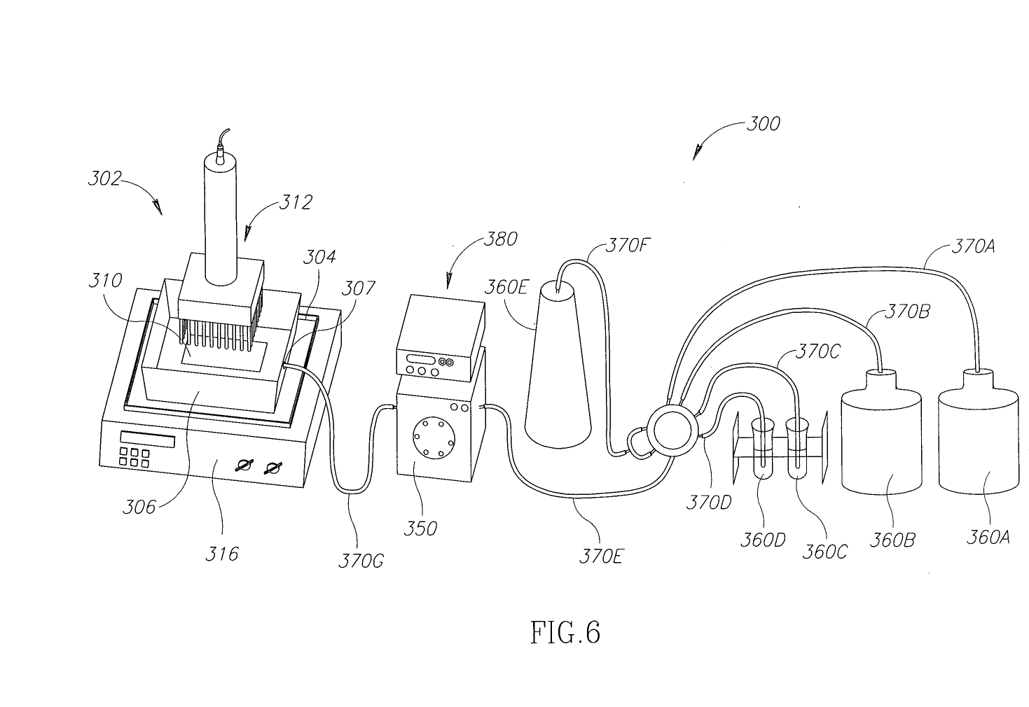

According to some embodiments, there are provided a device and system for the automatic and enhanced processing of a membrane having proteins associated therewith. The membrane may include, for example, a western blot immunoassay membrane having proteins electrotransferred thereto. For example, the membrane may include a nitrocellulose membrane, a PVDF membrane, a nylon membrane, and the like. Reference is now made to FIG. 6, which schematically illustrates a system for automatic and enhanced immunoassay processing, according to some embodiments. As shown in FIG. 6, the system (such as system 300) may include a device (302), for ultrasound-assisted enhanced processing of a membrane having proteins associated therewith. The device may include, for example, any of the device described hereinabove and any modifications thereof. For example, device 302 may include one or more shaking platforms (such as platform 304), which are attached to a motor (not shown). The shaking platform may move horizontally and/or vertically around an axis, at a constant speed, varying speed, or both, wherein the speed at which the shaking platform is moving may be in the range of, for example, 0 rpm to 1000 rpm and may be automatically and/or manually determined. On the top surface of the shaking platform one or more containers (such as container 306) may be placed. The container may be at any shape and size. The container may have rigid walls and may be constructed of plastic, glass, and the like. The container is constructed such that it may be used to hold/retain fluids. Optionally, container 306 may have one or more openings/aperture (such as aperture 307), which may be used to allow at will the removal or addition of fluids to the container. Optionally, container 306 may include a lid (not shown). Within container 306, a membrane having proteins associated therewith (such as membrane 310) may be placed. An ultrasonic transducer (such as transducer 312) is located such that ultrasonic energy produced by the transducer is directed towards container 306 and the membrane retained therein. The ultrasonic transducer may include any type of ultrasonic transducer. The ultrasonic transducer may include one or more oscillating elements and may produce ultrasonic energy at the range of about 1-1000 kHz, at a power of about 1-100 Watt. The ultrasonic transducer may be located at any vertical distance from the container. For example, the ultrasonic transducer may be located above the container. For example, the ultrasonic transducer may be located below the container. The ultrasonic transducer may be attached to the container lid. The ultrasonic transducer may be in direct contact with the fluids in the container. The ultrasonic transducer may be controlled by the ultrasonic transducer control unit, which is adapted to provide the ultrasonic transducer with the appropriate operating parameters (such as, for example, power, frequency, pulse length, and the like). In addition, the device may include a controller unit (such as controller unit 316), which may include a user input device (such as keypad, keyboard, dials, buttons, touch screen, and the like, or any combination thereof), a display, and the like. The controller unit may be used to control the operation of the device. For example, the controller unit may be used to set the speed, direction and length of time at which the shaking platform is moving. For example, the controller unit (316) may be used to control the ultrasonic transducer control unit. In addition, the system may include a one or more pumps (shown for example in FIG. 6, as one pump, 350) that may be used to transfer (add or remove) various fluids from between various locations. The pump may be unidirectional or bi-directional, that is, it may have direct and reverse modes that allow unidirectional or bi-directional transfer of fluids. The pump may include any pump such as, for example, but not limited to, a minipump, peristaltic pump, unidirectional pump, bi-directional pump, and the like. The system may further include one or more tanks (also referred to herein as flasks, vials and test tubes; shown for example, in FIG. 6 as tanks 360A-E), that may be connected directly or indirectly to another tank and/or to a pump (such as pump 350). In addition, tubes, pipes, rigid tubes, flexible tubes, rigid pipes, flexible pipes, and the like, (shown, for example in FIG. 6 as flexible tubes 370A-G), may be used to connect and/or transfer fluids between various constituents of system 300. The tubes may be constructed of various materials, such as, for example, plastic, rubber, glass, and the like. The tubes (such as tubes 370A-E) may be used to connect between the tanks (such as tanks 360A-E) and pump 350. For example, the tubes (such as, for example, tube 370G) may be used to connect between pump 350 and container 306. The tubes may be used for the transfer of fluids to and from various constituents of the system (300). The tanks (such as, tanks 360A-E) may be of various shapes and sizes and may be constructed of various materials, such as, for example, plastic, glass, metal, and the like. The tanks may hold various fluids, such as, for example, reagents, buffers, washing fluids, and the like. For example, the tanks (such as, for example vials 360C-D), may hold various antibody solutions (such as, for example, primary antibody, secondary antibody, polyclonal antibody, monoclonal antibody, conjugated antibody, and the like, or any combination thereof). For example, the tanks (such as, for example, flask 360A) may hold various buffer solutions (such as, for example, PBS, TBS, water, detergents, and the like, or combinations thereof). For example, the tanks (such as, for example, tank 360B) may hold various protein solutions (such as, for example, BSA solution, milk solution, enzyme solution, and the like, or combinations thereof). In some embodiments, the tanks (such as, for example, tank 360E), may be initially empty and may be used to collect and/or retain fluids (such as, for example, waste fluids) that may be transferred thereto. The tanks may have lids, faucets, electrically controlled faucets, opening, aperture, electrically controlled opening, electrically controlled aperture, and the like, that may allow the opening and/or closure of the tank. In some embodiments, the tank may be automatically filled, manually filled, or both. In some embodiments, the tank temperature may be controlled actively (by attaching a heat maintaining device, (such as, for example, a mini refrigerator or a mini heater) to the tank) and/or passively (for example, by placing the tank in a hot/cold fluid (water) bath, ice bucket, and the like). In some embodiments, fluids may be transferred (pumped), from the tank (such as, for example, tanks 360A-D) to the container (such as container 306), which holds membrane 310. In some embodiments, fluids may be transferred (pumped) from the container which holds membrane 310 (such as container 306) to the tanks (such as, for example, tank 360E). The transfer (pumping) to and/or from the tanks may be performed by pump (such as pump 350) and may be controlled automatically and/or manually. In addition, system 300 may further include a central control unit (such as control unit 380) that may be used to control, time and coordinate the operation of the system. The control unit may include various components, such as, for example, a central processing unit, a user interface (such as, for example, a keyboard, a key pad, a touch screen, and the like), a timer, an electronic controller, a display, an audible alarm component, and the like, or any combination thereof. For example, the central controlling unit may control the operation of the pump, such as, for example, the rate of pumping, the pumping direction, time length of pumping, order of pumping (that is, to/from which tank fluids are transferred). For example, the central control unit may control the operation of device 302, by controlling the controller unit (316) of the device. Controlling the control unit of the device may include directly or indirectly controlling the direction and speed of the shaking platform, length, power, frequency, and the like, of the ultrasonic energy produced by the ultrasonic transducer, and the like. In some embodiments, the central control unit may be programmed by the user, so as to predetermine the length of each step performed by the system, the identity of each step performed by the system (that is, what will the system do in each step), and the like.

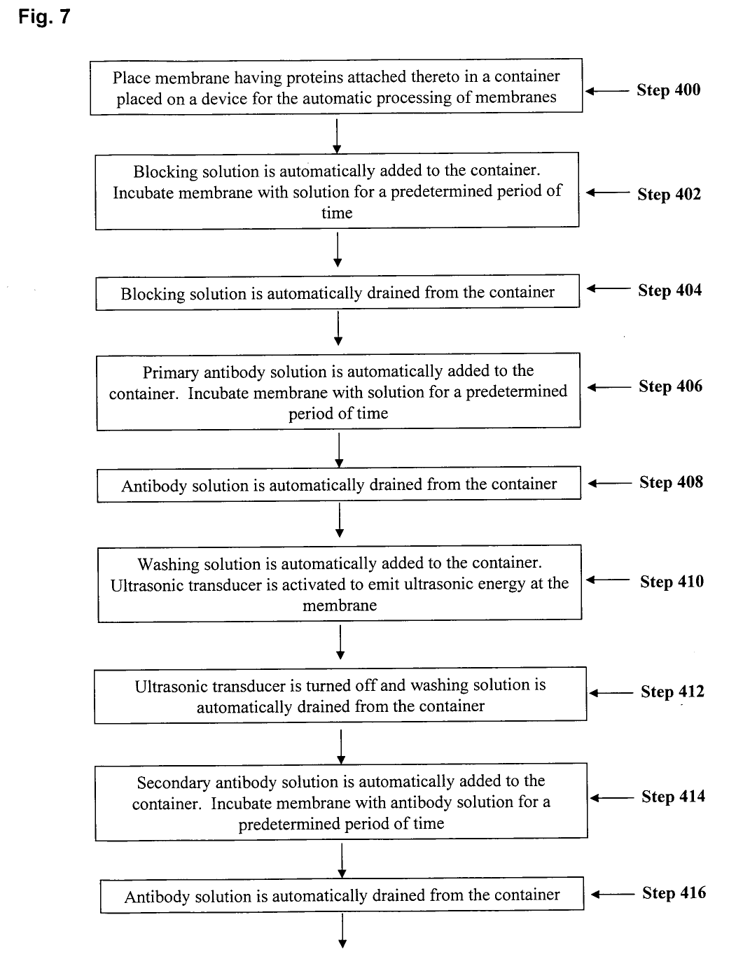

According to some embodiments, there is further provided a method for the ultrasound assisted automatic and enhanced processing of a membrane having proteins associated therewith. Reference is now made to FIG. 7, which illustrates a schematic flow chart of the steps of the method, according to some embodiments. As shown in FIG. 7, in Step 400, a membrane (having proteins associated therewith) is placed in a container (such as container 306 in FIG. 6), which is placed on a device (such as device 302 in FIG. 6). The membrane may include, for example, a nitrocellulose membrane, a PVDF membrane, a nylon membrane, and the like, having proteins electrotransferred thereto. For example, the membrane may include a western blot immunoassay membrane. In step 402, a predetermined amount of blocking solution (such as, for example, 10-20 ml of BSA solution) is transferred (pumped) from the appropriate tank (such as, for example, tank 360A in FIG. 6) to the container (such as, for example, container 306 of FIG. 6). The blocking solution may be left in the container for a predetermined period of time, while the container is shaking at a predetermined speed. The shaking of the container may be achieved by activating the shaking platform (such as, for example, shaking platform 304 in FIG. 6) to shake at a constant or variable speed. For example, the blocking solution may be left in the container for 30-60 minutes, while the shaking platform is shaking at a constant speed of 5-20 rpm. In step 404, after the predetermined incubation time of step 402 is ended, the blocking solution of step 402 is drained from the container to a collection tank, that may include, for example, a sink, a tank, a flask, a vial, a test tube, and the like. In some embodiments, draining of the container may be performed passively, by opening the container aperture (such as aperture 307 in FIG. 6) and letting the fluids drain by, for example, gravity force, to a collection/waste tank, such as, for example, a sink. In some embodiments, draining of the container may be performed actively, by opening the container aperture, and actively pumping the fluids from the container to a fluid collector (such as, for example, tank 360E in FIG. 6). In some embodiments, the opening of the container aperture is controlled electronically and automatically. Actively pumping the fluids may be performed, for example, by the pump (such as, for example, pump 350 in FIG. 6). Next, at step 406, a first antibody solution (primary antibody) may be transferred from the appropriate tank (such as, for example, tank 360C in FIG. 6) to the container. The first antibody solution may be left in the container for a predetermined period of time, while the container is shaking at a predetermined speed. The shaking of the container may be achieved by activating the shaking platform to shake at a constant or variable speed. For example, the first antibody solution (at an amount of, for example 2-20 ml at a dilution of 1:500-1:5000), may be left in the container for 10-60 minutes, while the shaking platform is shaking at a constant speed of 5-20 rpm. In step 408, after the predetermined incubation time of step 406 is ended, the first antibody solution is drained from the container. Draining of the first antibody solution may be passive or active. In some embodiments, the first antibody solution is drained to a collection tank, such as, for example, a sink, tank, flask, test tube, vials, and the like. In some embodiments, the first antibody solution is drained back to the same tank from which it was transferred (for example, tank 360C). In such an embodiment, the pump is operating in reverse mode to pump the first antibody solution from the container to the original tank. Such a mode of operation allows the collection and reuse of the first antibody solution and hence results in lowering the costs of the assay. Next, at step 410, a predetermined amount of washing solution (such as, for example, 10-20 ml of PBS buffer) is transferred (pumped) from the appropriate tank (such as, for example, tank 360B in FIG. 6) to the container. The washing solution may be left in the container for a predetermined period of time, while the container is shaking at a predetermined speed. In addition, ultrasonic energy, emitted from the ultrasonic transducer (such as ultrasonic transducer 312 in FIG. 6) is aimed at the membrane. The ultrasonic energy may be emitted, for example, for the entire length of step 410 or for any part of step 410. The shaking of the container may be achieved by activating the shaking platform to shake at a constant or variable speed. For example, the washing solution may be left in the container for 1-5 minutes, while the shaking platform is shaking at a constant speed of 5-20 rpm and ultrasonic energy is emitted at the frequency of about 1-1000 kHz, and power of about 1-100 Watt. In step 412, after the predetermined incubation time of step 410 is ended, the operation of the ultrasonic transducer is stopped, and the washing solution of step 410 is drained from the container to a collection tank, that may include, for example, a sink, a tank, a flask, a vial, a test tube, and the like. In some embodiments, draining of the container may be performed passively, or actively. Steps 410-412 may be repeated one or more times with different or identical parameters. The parameters may include such parameters as, but not limited to: length of the step, amount of washing fluid, the content of washing fluid, shaking speed, frequency, power and length of the ultrasonic energy, and the like, or any combination thereof. Next, at step 414, a second antibody solution may be transferred form the appropriate tank (such as, for example, tank 360D in FIG. 6) to the container. The second antibody solution may be left in the container for a predetermined period of time, while the container is shaking at a predetermined speed. The shaking of the container may be achieved by activating the shaking platform to shake at a constant or variable speed. For example, the second antibody solution (at an amount of, for example 2-20 ml at a dilution of 1:10000), may be left in the container for 10-60 minutes, while the shaking platform is shaking at a constant speed of 5-20 rpm. In step 416, after the predetermined incubation time of step 416 is ended, the second antibody solution of step 416 is drained from the container. Draining of the second antibody solution may be passive or active. In some embodiments, the second antibody solution is drained to a collection tank, such as, for example, a sink, tank, flask, test tube, vials, and the like. In some embodiments, the second antibody solution is drained back to the same tank from which it was transferred (for example, tank 360D in FIG. 6). Next, at step 418, a predetermined amount of washing solution (such as, for example, 10-20 ml of PBS buffer) is transferred (pumped) from the appropriate tank (such as, for example, tank 360B in FIG. 6) to the container. The washing solution may be left in the container for a predetermined period of time, while the container is shaking at a predetermined speed. In addition, ultrasonic energy, emitted from the ultrasonic transducer (such as ultrasonic transducer 312 in FIG. 6) is aimed at the membrane. The ultrasonic energy may be emitted, for example, for the entire length of step 418 or for any part of step 418. The shaking of the container may be achieved by activating the shaking platform to shake at a constant or variable speed. For example, the washing solution may be left in the container for 1-5 minutes, while the shaking platform is shaking at a constant speed of 5-20 rpm and ultrasonic energy is emitted at the frequency of about 1-100 kHz, and power of about 1-100 Watt. In step 420, after the predetermined incubation time of step 418 is ended, the operation of the ultrasonic transducer is stopped, and the washing solution of step 418 is drained from the container to a collection tank, that may include, for example, a sink, a tank, a flask, a vial, a test tube, and the like. In some embodiments, draining of the container may be performed passively, or actively. Steps 418-420 may be repeated one or more times with different or identical parameters. The parameters may include such parameters as, but not limited to: length of the step, amount of washing fluid, the content of washing fluid, shaking speed, frequency, power and length of the ultrasonic energy, and the like, or any combination thereof. At step 422, washing solution is added and left in the container and a visual and/or audible alarm is activated to indicate the user that the processing of the membrane has ended and that the membrane is ready for further manipulation. In some embodiments, the various steps of the method may be predetermined by the user, for example, by programming the central control unit to determine the various parameters of each step in the method.

In the description and claims of the application, each of the words “comprise” “include” and “have”, and forms thereof, are not necessarily limited to members in a list with which the words may be associated.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Ultrasound-Assisted Enhanced Western Blot Immunoassay

### Materials and Methods

Ultrasound generator: Sonics Vibrocell, Sonics&Materials Inc, with 96-wells adaptor for maximal covering of membrane area. Standard level of power, about 25 Watt; Frequency of 20 kHz, time of ultrasonic radiation: about 2-3 min.

Protein samples (Hela cell lysate) were run on a Polyacrylamide gel (PAGE-Gel) and transferred to a nitrocellulose membrane using the Bio-Rad Mini-Protean system according to the manufacturer's instructions.

Antibodies were purchased from Cell Signaling Technologies. Standard dilution of antibodies: 1:1000-1:2000 for primary antibody (against p100/p52), and 1:10000 for secondary antibody (HRP conjugated).

### Results

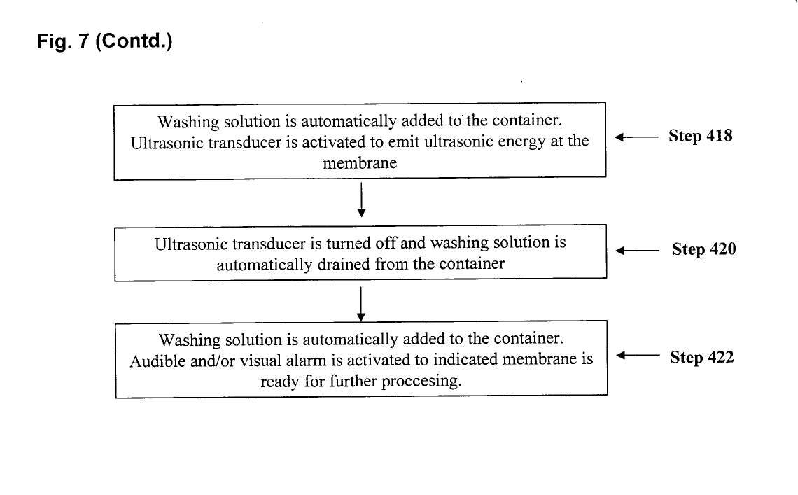

The effect of ultrasonic treatment at various steps of membrane processing (such as, blocking, incubation with antibodies, washing, and the like) was tested. Results are presented in FIG. 8, panels A, B and C. FIG. 8 panel A, is a pictogram of a developed membrane that was processed in the presence of ultrasonic energy for 1 hour during incubation of the nitrocellulose membranes with primary and secondary antibodies. FIG. 8 panel B, is a pictogram of a developed membrane that was processed in the presence of ultrasonic energy for 1 hour during incubation of the nitrocellulose membranes with secondary antibody. The results shown in FIG. 8 Panels A and B show that the ultrasound treatment does not effectively remove background, and moreover, decrease specificity of the antibodies. FIG. 8 panel C, is a pictogram of a developed membrane that was processed in the presence of ultrasonic energy during the washing steps (after first and second antibody incubation), 1-2 washing steps, 2-3 minutes each. The results presented in FIG. 8, panel C demonstrate that applying ultrasonic energy during the washing steps of the nitrocellulose membranes (after incubation with antibodies) yields enhanced results—usage of ultrasound treatment during the washing steps allow decreasing the washing time by at least 3 folds: instead of 3 washing steps (5-10 minutes each) after each antibody incubation in the absence of ultrasonic treatment, only 1-2 washing steps of 2-3 minutes in the presence of ultrasonic treatment, are needed to obtain at least the same effectiveness. Arrows indicate the specific bands of the p100 and p52 proteins in the Hela cells lysate.

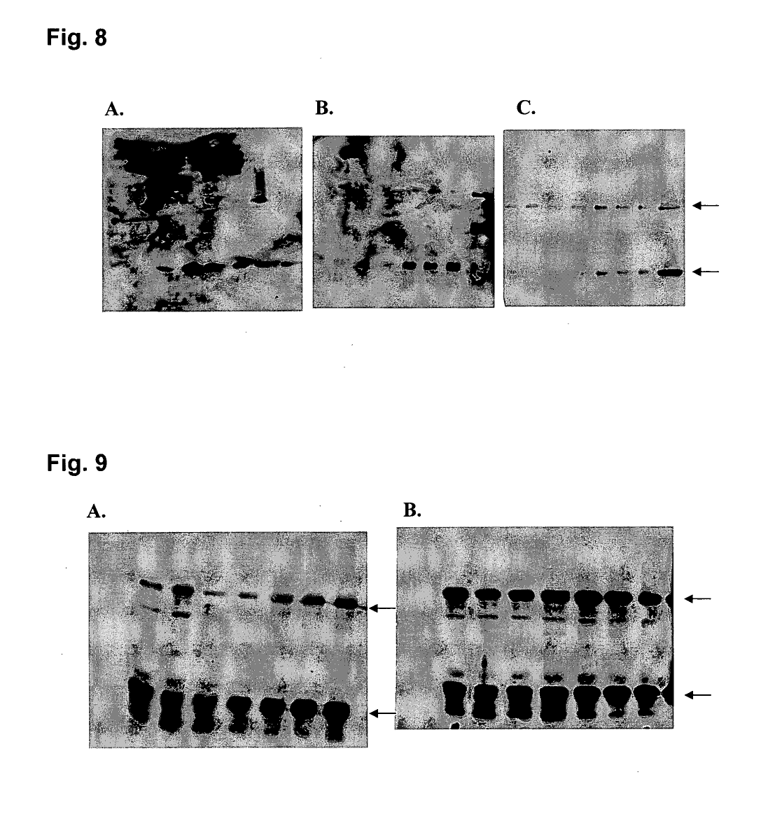

This is further demonstrated in FIG. 9, which shows two membranes, processed by a standard protocol (FIG. 9 panel B, “standard protocol”, detailed in Table 2) and by a protocol which involve the use of ultrasonic energy during the washing steps (FIG. 9, panel A). As shown in FIG. 9, usage of an “ultrasound assisted” protocol (detailed in Example 2, below), allows saving of at least 50-60% of processing time without compromising the quality of the results. Arrows indicate the specific bands of the p100 and p52 proteins in the Hela cells lysate.

### Example 2

### Ultrasound-Assisted Protocol for Processing a Membrane Having Proteins Associated Therewith

Listed in Table 1 are the main and general steps of a standard (routinely and generally used) protocol and of an “ultrasound-assisted” protocol (utilizing ultrasound at the washing steps). Both protocols are used for the processing of a membrane (such as nitrocellulose membrane, PVDF membrane, nylon membrane) having proteins associated therewith. As summarized in Table 1, the ultrasound assisted protocol is a faster, more accurate and better quality protocol as compared to the standard protocol.

TABLE 1

| | | “ultrasound- |
|---|---|---|
| Step | “Standard” protocol | assisted” protocol |
| 1 | Blocking buffer | Blocking buffer (30 min) |
| | (1 hour-overnight) | |
| 2 | Incubate with primary | Incubate with primary |
| | antibody 1 h-overnight | antibody 1 h |
| 3 | Wash 3 x 5-10 min | Wash 2 x 2 min with |
| | | ultrasound treatment |
| 4 | Incubate with secondary | Incubate with secondary |
| | antibody—45-60 min | antibody—30-45 min |
| 5 | Wash 3 x 5-10 min | Wash 3 x 2 min with |
| | | ultrasound treatment |
| 6 | Stripping after detection—5 min | Stripping after |
| | | detection—5 min |
| 7 | Neutralization by washing—5 min | Neutralization by |
| | | washing—5 min |
| 8 | Blocking buffer (go to step 1) | Blocking buffer—no need |
| | 1 h-overnight | |
| 9 | Primary antibody 1 h-overnight | Primary antibody |
| | | (go to step 2) 1 h |

| | |
|---|---|
| Minimum total time of first processing | Total time of first |
| cycle—about 4 hours | processing cycle—~2 h |
| Minimum processing time of every next | Processing time of |
| cycle (after stripping) about 4 hours | every next cycle |
| | (after stripping)—~1.5 h |

### Example 3

### Protocol of an Automatic Processing of a Membrane Having Proteins Associated Therewith

As in Example 1, Protein Gels were run and proteins were electrotransferred to a nitrocellulose membrane using the Bio-Rad Mini-Protean system, according to the manufacturer's instructions. Antibodies used were purchased from Cell Signaling Technologies. Standard dilution of antibodies: 1:1000-1:2000 for primary antibody, and 1:10000 for secondary antibody-HRP conjugate. Ultrasonic transducer emits ultrasonic energy at a power of about 25 Watt and frequency of 20 kHz.

The membrane is placed in a container in the system for the automatic and enhanced processing of a membrane having proteins associated therewith. The protocol of the automatic processing includes the following steps:

Step 1: Blocking—10 mL (Tank A, (3% BSA or 10% non-fat milk solution in PBS-T (PBS, 0.1% Tween-20)), time 30 min, shaking ON). Drain to sink.

Step 2: Primary Antibody—5 mL solution (anti-NF-kB2 antibody (rabbit source), 1:2000 dilution in PBS-T), (Tank C, time 30 min, shaking ON). Discharge back to tank C.

Step 3: Washing I—10 mL (PBS-T) (Tank B, time 2 min, shaking ON, ultrasonic transducer ON 20 kHz, 25 Watt). Drain to sink.

Step 4: Washing II—10 mL (PBS-T) (Tank B, time 2 min, shaking ON, ultrasonic transducer ON 20 kHz, 25 Watt). Drain to sink.

Step 5: Secondary Antibody—5 mL solution (HRP-conjugated anti-rabbit antibody, 1:10000 in PBS-T) (Tank D, time 30 min, shaking ON). Drain back to tank D.

Step 6: Washing I—10 mL (PBS-T) (Tank B, time 2 min, shaking ON, ultrasonic transducer ON 20 kHz, 25 Watt). Discharge to sink.

Step 7: Washing II—10 mL (PBS-T) (Tank B, time 2 min, shaking ON, ultrasonic transducer ON 20 kHz, 25 Watt). Discharge to sink.

Step 8: Washing III—10 mL (PBS-T) (Tank B, time 2 min, shaking ON, ultrasonic transducer ON 20 kHz, 25 Watt).

Step 9: STOP—Visual and audible alarm ON, shaking OFF, ultrasonic transducer OFF.

While the certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1. A method for enhancing immunoassay for identification of proteins, the method comprising: providing a membrane having proteins associated therewith, wherein the membrane is in contact with one or more antibody solutions; and
washing the one or more antibody solutions from the membrane in the presence of ultrasonic energy directed at the membrane;
thereby enhancing the immunoassay for the identification of proteins.

2. The method of claim 1, wherein the porous matrix comprises a polyacrylamide gel, an agarose gel, gelatin gel, or any combination thereof.

3. The method of claim 1, wherein the membrane is made of nitrocellulose membrane, polyvinylidene fluoride polyvinylidene difluoride (PVDF) membrane, nylon membrane, or any combination thereof.

4. The method of claim 1, wherein the antibody solution comprises a polyclonal antibody, a monoclonal antibody, serum, conjugated antibody, or any combination thereof.

5. (canceled)

6. The method of claim 1, further comprising repeating the washing one or more times at equal or different time intervals.

7. The method of claim 1, wherein the ultrasonic energy is applied by one or more ultrasonic transducers, adapted to emit ultrasonic energy at a frequency of about 1000 kHz or less.

8-16. (canceled)

17. A device for enhanced processing of a membrane having proteins associated therewith said device comprising:
a container situated on a platform, wherein the container is to retain a membrane having proteins associated therewith;
one or more ultrasonic transducers, configured to emit ultrasonic energy towards the container;
thereby enhancing processing of the membrane.

18. The device of claim 17, wherein the processing comprises: washing, incubating, shaking, rocking, developing, applying ultrasonic energy or any combination thereof.

19. The device of claim 17, wherein the membrane is made of nitrocellulose, polyvinylidene fluoride polyvinylidene difluoride (PVDF), nylon or any combination thereof.

20-21. (canceled)

22. The device of claim 17, further comprising a control unit comprising a user interface, a display, a key pad, an alarm, a dial, or any combination thereof.

23. The device of claim 17, wherein the platform is attached to a motor, and wherein the platform is configured to move horizontally and/or vertically around an axis, at constant speed, varying speed, or both.

24-26. (canceled)

27. The device of claim 17, wherein the one or more ultrasonic transducer are adapted to emit focused ultrasonic energy and/or surface ultrasonic energy, at a frequency of about 1000 kHz or less.

28. The device of claim 17, wherein said one or more ultrasonic transducers are located above the container, below the container, or both.

29-31. (canceled)

32. The device of claim 17, wherein at least one of said ultrasonic transducers comprises more than one oscillating elements.

33. A system for enhanced processing of a membrane having proteins associated therewith, said system comprising
a container situated on a platform, wherein the container is configured to retain a membrane having proteins associated therewith;
one or more ultrasonic transducers, configured to emit ultrasonic energy towards the container; and
a pump, configured to transfer fluid between the container and one or more tanks;
thereby enhancing processing the membrane.

34. The system of claim 33, wherein the processing comprises: washing, incubating, shaking, rocking, developing, applying ultrasonic energy or any combination thereof.

35. The system of claim 33, wherein the membrane is made of nitrocellulose, polyvinylidene fluoride polyvinylidene difluoride (PVDF), nylon or any combination thereof.

36. (canceled)

37. The system of claim 33, further comprising a central control unit, comprising a user interface, a display, a key pad, an alarm, a dial, or any combination thereof.

38-39. (canceled)

40. The system of claim 33, wherein the platform is adapted to move horizontally and/or vertically around an axis, at a constant speed and/or varying speed.

41-42. (canceled)

43. The system of claim 33, wherein the ultrasonic transducer is adapted to emit ultrasonic energy at a frequency of 1000 kHz or less.

44. The system of claim 33, wherein said one or more ultrasonic transducers are located above the container, below the container, or both.

45-47. (canceled)

48. The system of claim 33, wherein at least one of said one or more ultrasonic transducers comprises more than one oscillating elements.

49. The system of claim 33, wherein the pump comprises peristaltic pump, mini pump, unidirectional pump, bidirectional pump, or any combination thereof.

50-52. (canceled)

53. The method of claim 1, further comprising, prior to providing the membrane, separating the proteins by electrophoresis in a porous matrix; and
electophoretically transferring the proteins from the porous matrix to the membrane.
